SEQUENCE LISTING (29 sequences)

SEQ ID NO: 1 - protein, 18 aa, Artificial sequence
  Synthetic antimicrobial peptide
KNLRRXXRKXXHIIKKYG

SEQ ID NO: 2 - protein, 18 aa, Artificial sequence
  Synthetic antimicrobial peptide
KNLRRGIRKIIHIIKKYG

SEQ ID NO: 3 - protein, 18 aa, Artificial sequence
  Synthetic antimicrobial peptide
KNLRRTIRKIIHIIKKYG

SEQ ID NO: 4 - protein, 18 aa, Artificial sequence
  Synthetic antimicrobial peptide
KNLRRSIRKIIHIIKKYG

SEQ ID NO: 5 - protein, 18 aa, Artificial sequence
  Synthetic antimicrobial peptide
KNLRREIRKIIHIIKKYG

SEQ ID NO: 6 - protein, 18 aa, Artificial sequence
  Synthetic antimicrobial peptide
KNLRRDIRKIIHIIKKYG

SEQ ID NO: 7 - protein, 18 aa, Artificial sequence
  Synthetic antimicrobial peptide
KNLRRAIRKIIHIIKKYG

SEQ ID NO: 8 - protein, 18 aa, Artificial sequence
  Synthetic antimicrobial peptide
KNLRRIIRKIIHIIKKYG

SEQ ID NO: 9 - protein, 18 aa, Artificial sequence
  Synthetic antimicrobial peptide
KNLRRIGRKIIHIIKKYG

SEQ ID NO: 10 - protein, 18 aa, Artificial sequence
  Synthetic antimicrobial peptide
KNLRRITRKIIHIIKKYG

SEQ ID NO: 11 - protein, 18 aa, Artificial sequence
  Synthetic antimicrobial peptide
KNLRRISRKIIHIIKKYG

SEQ ID NO: 12 - protein, 18 aa, Artificial sequence
  Synthetic antimicrobial peptide
KNLRRIERKIIHIIKKYG

SEQ ID NO: 13 - protein, 18 aa, Artificial sequence
  Synthetic antimicrobial peptide
KNLRRIDRKIIHIIKKYG

SEQ ID NO: 14 - protein, 18 aa, Artificial sequence
  Synthetic antimicrobial peptide
KNLRRIARKIIHIIKKYG

SEQ ID NO: 15 - protein, 18 aa, Artificial sequence
  Synthetic antimicrobial peptide
KNLRRIIRKIIHIIKKYG

SEQ ID NO: 16 - protein, 18 aa, Artificial sequence
  Synthetic antimicrobial peptide
KNLRRIIRKGIHIIKKYG

SEQ ID NO: 17 - protein, 18 aa, Artificial sequence
  Synthetic antimicrobial peptide
KNLRRIIRKTIHIIKKYG

SEQ ID NO: 18 - protein, 18 aa, Artificial sequence
  Synthetic antimicrobial peptide
KNLRRIIRKSIHIIKKYG

SEQ ID NO: 19 - protein, 18 aa, Artificial sequence
  Synthetic antimicrobial peptide
KNLRRIIRKEIHIIKKYG

SEQ ID NO: 20 - protein, 18 aa, Artificial sequence
  Synthetic antimicrobial peptide
KNLRRIIRKDIHIIKKYG

SEQ ID NO: 21 - protein, 18 aa, Artificial sequence
  Synthetic antimicrobial peptide
KNLRRIIRKAIHIIKKYG

SEQ ID NO: 22 - protein, 18 aa, Artificial sequence
  Synthetic antimicrobial peptide
KNLRRIIRKIIHIIKKYG

SEQ ID NO: 23 - protein, 18 aa, Artificial sequence
  Synthetic antimicrobial peptide
KNLRRIIRKIGHIIKKYG

SEQ ID NO: 24 - protein, 18 aa, Artificial sequence
  Synthetic antimicrobial peptide
KNLRRIIRKITHIIKKYG

SEQ ID NO: 25 - protein, 18 aa, Artificial sequence
  Synthetic antimicrobial peptide
KNLRRIIRKISHIIKKYG

SEQ ID NO: 26 - protein, 18 aa, Artificial sequence
  Synthetic antimicrobial peptide
KNLRRIIRKIEHIIKKYG

SEQ ID NO: 27 - protein, 18 aa, Artificial sequence
  Synthetic antimicrobial peptide
KNLRRIIRKIDHIIKKYG

SEQ ID NO: 28 - protein, 18 aa, Artificial sequence
  Synthetic antimicrobial peptide
KNLRRIIRKIAHIIKKYG

SEQ ID NO: 29 - protein, 18 aa, Artificial sequence
  Synthetic antimicrobial peptide
KNLRRIIRKIIHIIKKYG

(19) United States
(12) Patent Application Publication
(10) Pub. No.: US 2005/0245452 A1
(43) Pub. Date: Nov. 3, 2005

(54) Pharmaceutical use of novispirins

(75) Inventor: Hans-Henrik Kristensen Hogenhaug, Soelleroed (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/092,496

(22) Filed: Mar. 29, 2005

Related U.S. Application Data
(60) Provisional application No. 60/559,817, filed on Apr. 5, 2004.
(60) Provisional application No. 60/606,476, filed on Sep. 1, 2004.

(30) Foreign Application Priority Data
Mar. 29, 2004 (DK) PA 2004 00503
Aug. 26, 2004 (DK) PA 2004 01288

(51) Int. Cl. A61K 38/16; C07K 7/08

(52) U.S. Cl. 514/13; 530/326

(57) ABSTRACT

The present invention relates to use of Novispirin antimicrobial polypeptides in preparation of a pharmaceutical composition suitable for use against microbial lung infections or skin infections or for use in promoting wound healing.

### CROSS-REFERENCE TO RELATED APPLICATIONS

This application claims priority or the benefit under 35 U.S.C. 119 of Danish application nos. PA 2004 00503 and PA 2004 01288 filed Mar. 29, 2004 and Aug. 26, 2004, respectively, and U.S. provisional application Nos. 60/559,817 and 60/606,476 filed Apr. 5, 2004 and Sep. 1, 2004, respectively, the contents of which are fully incorporated herein by reference.

### FIELD OF THE INVENTION

The present invention relates to pharmaceutical uses of novispirin antimicrobial polypeptides.

### BACKGROUND OF THE INVENTION

There is a continuing need for new antimicrobial agents, particularly those that are effective in killing pathogens resistant to conventional antibiotics. Specific treatments of interest include administration to the lungs of patients with microbial infections (e.g., pneumonia) caused, e.g., by P. aeruginosa, S. maltophilia, etc., and to forestall the emergence of resistance to other antibiotics. Other specific treatments of interest include application to the skin of patients with serious wounds, to promote the healing process of such wounds; or application to patients suffering from skin infections, such as acne.

Acne is a skin condition which has plugged pores (blackheads and whiteheads), inflamed pimples (pustules), and deeper lumps (nodules). Acne occurs on the face, as well as the neck, chest, back, shoulders, and upper arms. Although most teenagers get some form of acne, adults in their 20's, 30's, 40's, or even older, can develop acne. Untreated acne can leave permanent scars; to avoid acne scarring, treating acne is important.

Accordingly it is an object of the present invention to provide methods of using novispirin antimicrobial polypeptides for preparing medicaments suitable for use against lung infections, such as pneumonia or for promoting wound healing or treatment of skin infections, such as acne.

### SUMMARY OF THE INVENTION

In a first aspect the present invention relates to the use of an antimicrobial polypeptide for the manufacture of a pharmaceutical formulation for use against lung infections or skin infections or for use in promoting wound healing; wherein the antimicrobial polypeptide comprises the sequence
KNLRRX₁X₂RKX₃X₄HIIKKYG (SEQ ID NO: 1)
wherein X₁, X₂, X₃ and X₄ are independently selected from the group consisting of glycine, threonine, serine, glutamic acid, aspartic acid, isoleucine, D-alanine and D-isoleucine, provided that not more than 3 of the X residues are isoleucine; and a pharmaceutical acceptable carrier.

Embodiments of the present invention will be apparent from the below description and from the appended claims.

### DETAILED DESCRIPTION OF THE INVENTION

Novispirin Polypeptides

For use in the subject methods, any of the novispirins disclosed in PCT patent application WO 02/00839, modifications thereof, or a combination of one or more forms may be used. Novispirins include polypeptides of the formula

| | |
|---|---|
| KNLRRX₁X₂RKX₃X₄HIIKKYG | (SEQ ID NO:1) |

wherein X₁, X₂, X₃ and X₄ are independently selected from the group consisting of glycine, threonine, serine, glutamic acid, aspartic acid, isoleucine, D-alanine and D-isoleucine, provided that not more than three of the X residues are isoleucine. Preferred amino acids for the non-isoleucine residues are glycine, serine and threonine. Preferred are polypeptides wherein only one of X₁, X₂, X₃ and X₄ is selected from glycine, serine and threonine.

The sequence of the novispirin polypeptides may also be altered in various ways known in the art to generate targeted changes in sequence. The polypeptides will usually be substantially similar to the sequences provided herein, i.e., will differ by one amino acid, and may differ by two amino acids. The sequence changes may be substitutions, insertions or deletions.

The protein may be joined to a wide variety of other oligopeptides or proteins for a variety of purposes. By providing for expression of the subject peptides, various postranslational modifications may be achieved. For example, by employing the appropriate coding sequences, one may provide farnesylation or prenylation. In this situation, the peptide will be bound to a lipid group at a terminus, so as to be able to be bound to a lipid membrane, such as a liposome. In another example, the carboxy terminus of the peptide is amidated, thereby increasing the positive charge of the peptide.

The novispirins for use in the subject methods may be produced from eukaryotic or prokaryotic cells by recombinant methods, or may be synthesized in vitro as known in the art.

In one embodiment of the invention, the antimicrobial peptide consists essentially of the polypeptide sequence set forth in any one of SEQ ID NO: 2 to SEQ ID NO: 29, preferably the polypeptide sequence set forth in SEQ ID NO: 16.

The term “any one of SEQ ID NO: 2 to SEQ ID NO: 29” means SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29.

By “consisting essentially of” in the context of a polypeptide described herein, it is meant that the polypeptide is composed of the sequence set forth in the sequence listing, which sequence may be flanked by one or more amino acid or other residues that do not materially affect the basic characteristic(s) of the polypeptide.

Methods of Use

The present invention provides a method of use of an antimicrobial polypeptide for the manufacture of a pharmaceutical formulation (also referred to as a medicament) for use against lung infections or skin infections (such as acne, atopic dermatitis or seborrheic dermatitis, impetigo, folliculitis, furunculosis, carbunculosis, erysipelas, cellulitis) or for use in promoting wound healing; wherein the antimicrobial polypeptide comprises the sequence

| | |
|---|---|
| KNLRRX₁X₂RKX₃X₄HIIKKYG | (SEQ ID NO:1) |

wherein X₁, X₂, X₃ and X₄ are independently selected from the group consisting of glycine, threonine, serine, glutamic acid, aspartic acid, isoleucine, D-alanine and D-isoleucine, provided that not more than 3 of the X residues are isoleucine; and a pharmaceutical acceptable carrier.

The present invention also provides a method for treating lung infections or skin infections (such as acne, atopic dermatitis or seborrheic dermatitis, impetigo, folliculitis, furunculosis, carbunculosis, erysipelas, cellulitis) or for promoting wound healing with an antimicrobial polypeptide which comprises the sequence

| | |
|---|---|
| KNLRRX₁X₂RKX₃X₄HIIKKYG | (SEQ ID NO:1) |

wherein X₁, X₂, X₃ and X₄ are independently selected from the group consisting of glycine, threonine, serine, glutamic acid, aspartic acid, isoleucine, D-alanine and D-isoleucine, provided that not more than 3 of the X residues are isoleucine.

Formulations of novispirins may be administered to a host suffering from or predisposed to a microbial lung infection, such as pneumonia; preferably the lung infection is a bacterial lung infection, e.g., caused by gram negative bacteria.

Formulations of novispirins may also be administered to a host suffering from or predisposed to a microbial wound infection, preferably the infection is a bacterial infection, e.g., caused by gram negative bacteria.

Formulations of novispirins may also be administered to a host suffering from or predisposed to a skin infection, such as acne, atopic dermatitis or seborrheic dermatitis, impetigo, folliculitis, furunculosis, carbunculosis, erysipelas or cellulitis; preferably the skin infection is a bacterial skin infection, e.g., caused by Staphylococcus epidermidis, Staphylococcus aureus, Propionibacterium acnes, Pityrosporum ovale or Malassezia furfur.

Administration of the formulation may be topical, localized or systemic, depending on the specific microorganism, preferably it will be localized. Generally the dose of novispirin will be sufficient to decrease the microbial population by at least about 50%, usually by at least 1 log, and may be by 2 or more logs of killing. The compounds of the present invention are administered at a dosage that reduces the microbial population while minimizing any side-effects. It is contemplated that the composition will be obtained and used under the guidance of a physician for in vivo use.

The susceptibility of a particular microbe to killing with novispirins may be determined by in vitro testing. Typically a culture of the microbe is combined with novispirins at varying concentrations for a period of time sufficient to allow the protein to act, usually between about one hour and one day. The viable microbes are then counted, and the level of killing determined.

Microbes of interest include, but are not limited to, Gram-negative bacteria. Other bacteria of interest include, but are not limited to, Legionella sp., e.g., L. pneumophila; Mycoplasma sp., e.g., M. hominis, M. pneumonie; Mycobacterium sp., e.g., M. tuberculosis, M. leprae. Other bacteria of interest include Staphylococcus epidermidis, Staphylococcus aureus and Propionibacterium acnes.

Non-bacterial pathogens of interest include, but are not limited to, Pityrosporum ova/e, Malassezia furfur, Candida albicans, Trichophyton mentagrophytes and Epdidermophyton floccosum.

Various methods for administration may be employed. The polypeptide formulation may be given orally, or may be injected intravascularly, subcutaneously, peritoneally, by aerosol, opthalmically, topically, etc. For example, methods of administration by inhalation are well-known in the art. The dosage of the therapeutic formulation will vary widely, depending on the specific novispirin to be administered, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered once or several times daily, semi-weekly, etc. to maintain an effective dosage level. In many cases, oral administration will require a higher dose than if administered intravenously. The amide bonds, as well as the amino and carboxy termini, may be modified for greater stability on oral administration. For example, the carboxy terminus may be amidated.

Formulations

The novispirin polypeptides can be incorporated into a variety of pharmaceutical formulations (medicaments) for therapeutic administration. More particularly, the compounds of the present invention can be formulated into pharmaceutical compositions by combination with appropriate, pharmaceutical acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols.

As such, administration of the compounds can be achieved in various ways, including oral, buccal, rectal, parenteral, intraperitoneal, intradermal, transdermal, intracheal, etc., administration.

In one embodiment, a formulation for topical use comprises a cheating agent that decreases the effective concentration of divalent cations, particularly calcium and magnesium.

For example, agents such as citrate, EGTA or EDTA may be included, where citrate is preferred. The concentration of citrate will usually be from about 1 to 10 mM.

The compounds of the present invention can be administered alone, in combination with each other, or they can be used in combination with other known compounds (e.g., perforin, anti-inflammatory agents, antibiotics, etc.). In pharmaceutical dosage forms, the compounds may be administered in the form of their pharmaceutical acceptable salts. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with the compound in the host.

The pharmaceutical acceptable excipients, such as vehicles, adjuvants, carriers or diluents, are readily available to the public. Moreover, pharmaceutical acceptable auxiliary substances, such as pH adjusting and buffering agents, tonicity adjusting agents, stabilizers, wetting agents and the like, are readily available to the public.

Typical dosages for systemic administration range from 0.1 micrograms to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine.

The remaining lipid will normally be neutral or acidic lipids, such as cholesterol, phosphatidyl serine, phosphatidyl glycerol, and the like.

For preparing the liposomes, the procedure described by Kato et al. (1991) J. Biol. Chem. 266:3361 may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, novispirins may be formulated with other pharmaceutical active agents, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a novispirins formulation, e.g., interferon gamma, tumor necrosis factor alpha, interleukin 12, etc.

Synthesis of Novispirins

The subject peptides may be prepared by in vitro synthesis, using conventional methods as known in the art. Various commercial synthetic apparatuses are available, for example automated synthesizers by Applied Biosystems Inc., Foster City, Calif., Beckman, etc. By using synthesizers, naturally occurring amino acids may be substituted with unnatural amino acids, particularly D isomers, e.g., D-alanine and D-isoleucine, diastereoisomers, side chains having different lengths or functionalities, and the like. The particular sequence and the manner of preparation will be determined by convenience, economics, purity required, and the like.

Chemical linking may be provided to various peptides or proteins comprising convenient functionalities for bonding, such as amino groups for amide or substituted amine formation, e.g., reductive amination, thiol groups for thioether or disulfide formation, carboxyl groups for amide formation, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gelelectrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The present invention is further described by the following examples which should not be construed as limiting the scope of the invention.

### EXAMPLES

Chemicals used as buffers and substrates were commercial products of at least reagent grade.

### Example 1

Effect of Novispirin G10 (SEQ ID NO: 16) on a Pseudomonas aeruginosa Lung Infection in Rats

The bacterial strain, Pseudomonas aeruginosa, used in the study is CF86/57388A (1997), which is isolated from a cystic fibrosis patient and keeps stable mucoid phenotype. The rat strain used in the study is Lewis rat (7 weeks old, female) imported from Germany.

The amino acid sequence of Novispirin G10 is shown in SEQ ID NO: 16.

Both bacterial challenge and drug administration were done intratracheally. All rats were challenged with mucoid P. aeruginosa in alginate. The concentration of the bacteria was 1×10¹⁰ CFU/ml and each rat received 0.1 ml (1×10⁹ CFU) bacteria. Control group received placebo (sterile saline, 0.1 ml/rat) twice at hour 0 and 3, and the Novispirin G10 group received the peptide (0.1 mg/ml, 0.1 ml/rat) twice at hour 0 and 3. The animals were sacrificed on day 3, 5, 7 and 10 after challenge for parameters evaluation.

One to two rats died in each group after bacterial challenge. There is no difference between the two groups.

Macroscopic Pathology

Description of Gross Lung Pathology

On day 3, large area of lung consolidation was seen in the placebo control group, whereas in the Novispirin G10 treated group, the consolidation area was much smaller. On days 5, 7 and 10, large lung consolidation or abscess with adhesion, and atelectasis were seen in the control group mainly; and in the Novispirin G10 treated group, the pathology is dominated by small lung atelectasis, no abscess and adhesion found.

Apparently, the animals treated with Novispirin G10 exhibited significantly milder lung pathology in all 4 time points compared with the placebo control group.

Luna Pathological Scores

On day 3, no significant difference in the severity of lung scoring between the two groups. However, from day 5 on, Novispirin G10 treated mice showed significantly milder lung pathology compared with the control group (see the table below).

| Groups | Score 1 + 2 | Score 3 | Score 4 |
|---|---|---|---|
| Novispirin | 0/14 = 0 | 3/14 = 21.4% | 11/14 = 78.6% |
| G10 day 3 | | | |
| Control day 3 | 0/14 = 0 | 0/14 = 0 | 14/14 = 100% |
| P values | 1 | >0.25 | >0.25 |
| Novispirin | 0/14 = 0 | 10/14 = 71.4% | 0/14 = 0 |
| G10 day 5 | | | |
| Control day 5 | 0/14 = 0 | 0/14 = 0 | 14/14 = 100% |
| P values | 1 | <0.001 | <0.001 |
| Novispirin | 6/11 = 54.5% | 5/11 = 45.5% | 0/11 = 0 |
| G10 day 7 | | | |
| Control day 7 | 0/11 = 0 | 3/11 = 27.3% | 7/11 = 63.6% |
| P values | <0.005 | >0.25 | <0.005 |
| Novispirin | 11/14 = 78.6% | 3/14 = 21.4% | 0/14 = 0 |
| G10 day 10 | | | |
| Control day 10 | 1/12 = 8.3% | 4/12 = 33.3% | 7/12 = 58.3% |
| P values | <0.001 | >0.25 | <0.001 |

Histopathology of Lungs

On day 3, polymorphonuclear leukocyte infiltration in the lung tissues or bronchia could be seen mostly in the control group; whereas in the Novispirin G10 treated group, lung consolidation with chronic inflammation is the dominant change. On day 5, lung edema could be seen in the control group and milder chronic inflammation was seen in the Novispirin G10 treated group. On day 10, the lung pathology was tended to be cured in the Novispirin G10 treated group, however, in the control group, pulmonary edema could still be seen in some animal lungs.

The results of lung histopathology show that animals treated with Novispirin G10 intratracheally result in a milder lung inflammation and recover remarkably faster in the comparison with the placebo control group.

Lung bacteriology: Percentage of animals that cleared the bacteria in the lungs

| Treatment | Day 3 | | Day 5 | | Day 7 | | Day 10 | |
|---|---|---|---|---|---|---|---|---|
| Novispirin G10 group | 3/10 | (30%) | 5/5 | (50%) | 9/10 | (90%) | 10/10 | (100%) |
| Placebo group | 0/10 | (0) | 0/10 | (0) | 6/10 | (60%) | 6/9 | (66.67%) |

| | | | | |
|---|---|---|---|---|
| P value | >0.1 | <0.03 | >0.1 | <0.05 |

The results of lung bacteriology indicate that Novispirin G10 treatment significantly enhanced the lung bacteria clearance, which can be seen especially on day 3 and day 5 when compared with the control group. Faster lung bacterial clearance leads to quicker recovery of lung pathology.

Conclusion—Part 1

Intratracheal administration of Novispirin G10 in the concentration of 0.1 mg/ml after the establishment of a mucoid P. aeruginosa lung infection showed a significant effect on accelerating the lung bacterial clearance and reducing the severity of lung pathology. This would help to reduce the lung tissue damage and keep a better lung function in patients with P. aeruginosa pneumonia. Clearly, the milder lung pathology seen in the Novispirin G10 treated animals are associated closely with the faster bacterial clearance due to the administration of Novispirin G10 intratracheally in those animals.

Effects of Novispirin G10 Treatment on Lung Cytokine Response

Supernatants of the lungs were harvested from days 3, 5 and 7 after lung infection and treatment. Interferon-gamma (IFN-γ), tumor necrosis factor-alpha (TNF-α), interleukin4 (IL4), IL-6 and IL-10 were measured by using ELISA kits (Nordic BioSite, Sweden).

Lung cytokine responses (Mean + SD)

| Cyto- | | | | |
|---|---|---|---|---|
| kines | Treatment | Day 3 | Day 5 | Day 7 |
| IFN- | Novispirin | 65.3 ± 28.6 | 116.6 ± 42.1 | 79.9 ± 16.2 |
| gamma | G10 | | | |
| | Control | 115.9 ± 43.0 | 114.1 ± 11.9 | 87.0 ± 26.2 |
| TNF- | Novispirin | 159.2 ± 23.8 | 128.1 ± 19.0 | 121.8 ± 19.6 |
| alpha | G10 | | | |
| | Control | 219.2 ± 48.8 | 178.5 ± 38.1 | 140.9 ± 36.8 |
| IL-4 | Novispirin | 34.5 ± 4.5 | 45.3 ± 5.9 | 37.3 ± 5.9 |
| | G10 | | | |
| | Control | 42.5 ± 8.4 | 48.0 ± 4.5 | 52.9 ± 39.0 |
| IL-6 | Novispirin | 74.1 ± 10.0 | 65.2 ± 11.1 | 61.5 ± 11.0 |
| | G10 | | | |
| | Control | 91.1 ± 23.4 | 67.0 ± 11.7 | 56.9 ± 13.0 |
| IL-10 | Novispirin | 51.7 ± 2.6 | 57.8 ± 7.6 | 51.3 ± 2.9 |
| | G10 | | | |
| | Control | 70.5 ± 6.8 | 62.9 ± 8.5 | 53.7 ± 9.0 |

Discussion and Conclusion—Part 2

The results indicate that on day 3, the early phase of the infection, all 5 kinds of cytokine levels are found significantly lower in the Novispirin G10 treated group compared to the control group (see the table). The weaker cytokine responses in the early phase of infection might be correlated to the significantly lower lung bacteriology due to the direct bactericidal effect of the peptides. In the same time (day 3), the lung bacteriology was 175 times lower and the lung pathology was remarkably milder in the Novispirin G10 treated group compared to the control group. Lower bacterial load might lead to weaker immune responses against the bacterial lung infection, which could be explained at least partly by milder lung inflammatory responses and weaker cytokine responses seen in the Novispirin G10 treated group. The higher cytokine responses in the control group are non-specific since all 5 cytokines are higher in the same time-point of early infection, no matter Th1 or Th2 cytokines. With the time being, due to the decrease of bacterial load in the control group, the immune responses became less strong and the cytokine responses between the two groups become not significant.

In conclusion, Novispirin G10 treatment could clear the bacterial lung infection effectively via its direct bactericidal effect without strong immune responses, which might be helpful to diminish the tissue damage in lungs

### Example 2

Effect of Novispirin G10 on a Pseudomonas aeruginosa Wound Infection in Pigs

The bacterial strain, Pseudomonas aeruginosa, used in the study was ATCC 27853. The amino acid sequence of Novispirin G10 is shown in SEQ ID NO: 16.

Novispirin G10 was diluted in 0.01% acetic acid containing 0.1% BSA with a final concentration of 1 mg (substrate)/ml (solution); 100 micrograms/ml and 10 micrograms/ml. The carrier control was 0.01% acetic acid containing 0.1% BSA.

Method

Wound Chambers

The chambers are made out of titanium, have a screw top cover (also made out of titanium) and have an inner diameter of 25 mm.

Animal

After providing permission to conduct experiments with vertebrates in compliance with German animal protection laws, Göttinger minipigs (Fa. Ellegaard, Dalmose, Denmark, male, No. 86242 and 86260, 12 months old, 35 kg weight) were chosen to implant the chambers on. The pigs were kept at least 2 weeks in the stable, fed a standard porcine diet and housed at 20° C. to 30° C. in an atmosphere of approximately 65% humidity with a light cycle of 12 hours on and 12 hours off. The pig used for this study appeared during the experiments clinically healthy—rectal temperature and body weight remained normal over the observed period.

Wounding Procedure

For premedication and sedation 1 mg Midazolam/kg, 10 mg Ketamin/kg and 0.05 mg Atropin/kg were applied. The hair was shaved and abscised with depilatory cream (Veet, Reckitt Benckiser, Mannheim, Germany) afterwards the pig was washed with water and soap. For operation initiation 2 mg Propofol/kg was applied. The inhalational anesthesia was perpetuated during the implantation with 1% to 1.5% Isofluran and 30% O₂. Operative 0.75 mg Fentanyl was applied intramuscular.

Before wounding the skin was surgically prepared, washed with soap and desinfected with Octenisept (S&M, Norderstedt, Germany) for five minutes. Twelve wound chambers were implanted on the pigs back and flank between the base of the scapula and the iliac crest. The wounds were spaced a minimum of 5 cm apart and were created in four rows parallel to the columna vertebralis, two rows on each side of the columna vertebralis. The exact size and shape of the wounds were ensured by a special custom-made “round-scalpel”, which has a diameter of 33 mm. Epidermal, dermal subcutaneous tissue was removed, possible bleedings stanched by bipolar diathermy. The chambers were placed with an additionally secant v-incision at positions 6 respectively 12 o'clock on the musculus thoracicus longus fascia and the muscle fascia of the external ripcage muscles. The incisions were sutured with 4-0 Prolene followed by the closure of the chambers. Povidone-iodine salve (Betaisodonna-salve, Mundipharma, Austria) was smothered on the wound edges. The chambers were dressed with paraffin gauze (Jelonet, Smith & Nephew, Lohfelden, Germany) and sterile compresses (Johnson & Johnson, Gargrave, UK). Dressings were changed every second day under sedation (as described above).

For post operation analgesia, 0.9 mg Buprenorphin (equivalent 3 ml Temgesic, Essex Pharma, Munich, Germany) was applied. After terminating the experiment, the animals were euthanized by an intraveneous injection of T61 (Bayer, Leverkusen, Germany) at a dose of 1 ml/5 kg body weight.

Bacterial Background of each chamber was measured every second day to detect potential contamination. One contaminated chamber was detected. Eight days post operation all chambers were inoculated with bacteria.

Bacteria

For bacteria progeny, Pseudomonas aeruginosa (ATCC 27853) was plated on LB-Agar plates and incubated at 37° C. for 18 hours. One colony was picked, and transferred into 3 ml LB-Medium and grown over night at 37° C. at 300 rpm. The over night culture was diluted in 50 ml fresh LB-Medium and incubated for 2.5 hours at 37° C. at 200 rpm.

Bacteria were centrifuged at 4000 rpm, 5 min (Mergafuge 1.0 R, Heraeus, Hanau, Germany), and the pellet was resuspended in PBS. The amount of containing bacteria was determined by OD_{600 nm}-measurement and calculated as follows:
Colony forming Unit/ml=OD_{600 nm}×2.5×10⁸ (bacteria)

To inoculate one chamber, 5×10⁸CFU were used.

Time Course

The establishment of the infection was proven by the measurment of bacterial counts per chamber. All inoculated chambers were infected after two days.

At fourth day post infection, the antimicrobial effect of the testing substrate was measured in a time course of 4 hours in duration. Each chamber was cleaned before starting the time course, wound fluid was selected and blood clots were discarded. Before starting the time course each chamber was inoculated again with 1×10⁸ CFU Pseudomonas aeruginosa to get an equal bacterial amount for each chamber. One ml of the prepared samples with diluted test substrate and carrier control were applied in selected chambers.

Each chamber was rinsed with the applied solution several times. 50 microliters were taken to define the first value after 3 min for each chamber. Further on 50 microliters of chamber fluid were taken every hour until time course ends after 4 hours.

Samples were diluted in PBS (1:10; 1:100; 1:1000) and 10 microliters of each dilution and each sample was plated in triplicate on Pseudomonas isolation agar (Becton Dickinson, Heidelberg, Germany) for recovery of Pseudomonas aeruginosa and to discriminate further bacteria, respectively. After growth (for 18 hours at 37° C.) and counting of the bacterial colonies, the amount of colony forming units (CFU) was calculated per ml sample volume.

Mean for each testing solution

| Application | 0.05 hours | 1 hour | 2 hours | 3 hours | 4 hours |
|---|---|---|---|---|---|
| 1 mg | 667 | 1933 | 1167 | 517 | 733 |
| Novispirin G10 | | | | | |
| 100 micrograms | 7383 | 138181 | 167413 | 11661 | 2467 |
| Novispirin G10 | | | | | |
| 10 micrograms | 413869 | 1225733 | 34811 | 9612 | 17322 |
| Novispirin G10 | | | | | |
| Carrier control | 8263333 | 4048000 | 380025 | 14813 | 8431 |

Standard error for each testing solution

| Application | 0.05 hours | 1 hour | 2 hours | 3 hours | 4 hours |
|---|---|---|---|---|---|
| 1 mg | 0 | 933 | 357 | 314 | 528 |
| Novispirin G10 | | | | | |
| 100 micrograms | 3544 | 41153 | 52537 | 4023 | 1517 |
| Novispirin G10 | | | | | |
| 10 micrograms | 287566 | 139869 | 7287 | 3907 | 10220 |
| Novispirin G10 | | | | | |
| Carrier control | 1351612 | 383597 | 56445 | 11247 | 6841 |

### Example 3

Effect of Proline-Novispirin G10 on a Staphylococcus aureus Wound Infection in Pigs

The bacterial strain, Staphylococcus aureus, used in the study was ATCC 25923. The amino acid sequence of Novispirin G10 is shown in SEQ ID NO: 16.

Novispirin G10 was diluted in 0.01% acetic acid containing 0.1% BSA with a final concentration of 1 mg (substrate)/ml (solution); 100 micrograms/ml; 10 micrograms/ml and 1 microgram/ml. The carrier control was 0.01% acetic acid containing 0.1% BSA. As a positive control was used Synthetic Protegrin 1 (Charité, Institut f. Biochemie d. Humboldt Universität Berlin) diluted in 0.01% acetic acid containing 0.1% BSA with a final concentration of 100 micrograms/ml.

Method

Wound Chambers

The chambers are made of one piece of titanium, have a screw top cover (also made of titanium) and were built in teamwork with Cranio Construct Bochum. The chambers have a height of 14 mm, an inner diameter of 25 mm and an outer diameter of 32 mm without baseplate (with baseplate: 50 mm).

Animals

After providing permission to conduct experiments with vertebrates in compliance with German animal protection laws, Göttinger minipigs (Fa. Ellegaard, Dalmose, Denmark; female, 6 months old, 25-30 kg weight) were chosen to implant the chambers on. The pigs were kept at least 2 weeks in the stable, fed a standard porcine diet and housed at 20° C. to 30° C. in an atmosphere of approximately 65% humidity with a light cycle of 12 hours on and 12 hours off. The animals used for this study appeared, during the experiments, clinically healthy—rectal temperature and body weight remained normal over the observed period.

Wounding Procedure

For premedication and sedation 1 mg Midazolam/kg, 10 mg Ketamin/kg and 0.05 mg Atropin/kg were applied. The hair was shaved and abscised with depilatory cream (Veet, Reckitt Benckiser, Mannheim, Germany), afterwards the pig was washed with water and soap. For operation initiation 2 mg Propofol/kg was applied. The inhalational anesthesia was perpetuated during the implantation with 1% to 1.5% Isofluran and 30% O₂. Operative 0.75 mg Fentanyl were applied intramuscular.

Before wounding the skin was surgically prepared, washed with soap and disinfected with Octenisept (S&M, Norderstedt, Germany) for five minutes. Twelve BO-Chambers were implanted on the pigs back and flank between the base of the scapula and the iliac crest. The wounds were spaced a minimum of 5 cm apart and were created in four rows parallel to the columna vertebralis, two rows on each side of the columna vertebrtalis. The exact size and shape of the wounds were ensured by a special custom-made “round-scalpel” (also built in teamwork with Cranio Construct Bochum), which has a diameter of 33 mm. Epidermal, dermal subcutaneous tissues were removed, possible bleedings stanched by bipolar diathermy. The chambers were placed with an additionally secant v-incision at positions 6 respectively 12 o'clock on the musculus thoracicus longus fascia and the muscle fascia of the external ripcage muscles. The incisions were sutured with 2/0 Monocryl followed by the closure of the chambers. Povidone-iodine salve (Betaisodonna-salve, Mundipharma, Austria) was smothered on the wound edges. The chambers were dressed with paraffin gauze (Jelonet, Smith & Nephew, Lohfelden, Germany) and sterile compresses. Dressings were changed every second day under sedation (as described above).

For post operation analgesia, 0.9 mg Buprenorphin (equivalent 3 ml Temgesic, Essex Pharma, Munich, Germany) were applied. After terminating the experiment, the animals were euthanized by an intraveneous injection of T61 (Bayer, Leverkusen, Germany) at a dose of 1 ml/5 kg body weight.

Bacterial Background of each chamber was measured every second day, to detect potential contamination. Only one contaminated chamber was detected. Seven days post operation ten chambers were inoculated with bacteria.

Bacteria

For bacteria progeny Staphylococcus aureus (ATCC 25923) was plated on LB-Agar plate and incubated at 37° C. for 18 hours. One colony was picked and transfered into 3 ml LB-Medium for over night culture at 37° C. at 300 rpm. Over night culture was diluted in 50 ml fresh LB-Medium and incubated for 2.5 hours at 37° C. at 200 rpm.

Bacteria were pelleted, 4000 rpm, 5 min, Mergafuge 1.0 R (Heraeus, Hanau, Germany), and resuspended in PBS. Amount of containg bacteria was detremined by OD_{600 nm}-measurement and calculated as follows:
Colony forming Unit/ml=OD_{600 nm}×2.5×10⁸ (bacteria)

To inoculate one chamber 5×10⁸ were used.

Time Course

The establishment of the infection was proven by the measurement of bacterial counts per chamber. All inoculated chambers were infected after two days. Chamber No. 2 keeps bacteria free.

At day four after infection the antimicrobial effect of the testing substrate was measured in a time course of 4 hours in duration. Each chamber was cleaned before starting the time course, wound fluid was selected, blood clots were discarded. One ml of the prepared samples with diluted test substrate, positive control and carrier control were applied in selected chambers.

Each chamber was rinsed with the applied solution several times. 100 microliters were taken to define zero value for each chamber. Further 100 microliters of chamber fluid were taken every hour until time course ends after 4 hours.

Samples were diluted in PBS (1:10; 1:100; 1:1000) and 10 microliters of each dilution and the sample itself was plated in triplicate on Mueller-Hinton, 5% sheep blood agar (Becton Dickinson, Heidelberg, Germany) for recovery of staphylococci and to descriminate further bacteria, respectively. After growth (for 18 hours at 37° C.) and counting of the bacterial colonies, the number of colony forming units (CFU) was calculated per ml sample volume.

Mean for each testing solution:

| Application | 0 hours | 1 hour | 2 hours | 3 hours | 4 hours |
|---|---|---|---|---|---|
| control | 13900000 | 11500000 | 12300000 | 8166667 | 9900000 |
| infection | | | | | |
| blank value | 0 | 0 | 0 | 0 | 0 |
| carrier | 7750000 | 8250000 | 7583333 | 3231250 | 2066667 |
| control | | | | | |
| Protegrin 1 | 13200000 | 88667 | 12800 | 3525 | 129000 |
| [100 micro- | | | | | |
| grams/ml] | | | | | |
| Novispirin | 10700000 | 2817 | 2100 | 833 | 1233 |
| G10 | | | | | |
| [1 mg/ml] | | | | | |
| Novispirin | 1330000 | 790033 | 195125 | 142765 | 8467 |
| G10 | | | | | |
| [100 micro- | | | | | |
| grams/ml] | | | | | |
| Novispirin | 5065000 | 440500 | 304500 | 40910 | 1563188 |
| G10 | | | | | |
| [10 micro- | | | | | |
| grams/ml] | | | | | |
| Novispirin | 9060000 | 2508333 | 159067 | 33858 | 148333 |
| G10 | | | | | |
| [1 micro- | | | | | |
| gram/ml] | | | | | |

Standard error for each testing solution:

| Application | 0 hours | 1 hour | 2 hours | 3 hours | 4 hours |
|---|---|---|---|---|---|
| Control | 1078579 | 458258 | 971253 | 352767 | 1021437 |
| infection | | | | | |
| blank value | 0 | 0 | 0 | 0 | 0 |
| carrier control | 368578 | 1029193 | 1029193 | 346905 | 550124 |
| Protegrin 1 | 808290 | 2848 | 841 | 485 | 31198 |
| [100 micro- | | | | | |
| grams/ml] | | | | | |
| Novispirin | 550757 | 398 | 451 | 33 | 348 |
| [1 mg/ml] | | | | | |
| Novispirin | 75738 | 21232 | 21232 | 55724 | 1703 |
| [100 micro- | | | | | |
| grams/ml] | | | | | |
| Novispirin | 147046 | 21321 | 44751 | 7882 | 619703 |
| [10 micro- | | | | | |
| grams/ml] | | | | | |
| Novispirin | 245713 | 143572 | 21725 | 5083 | 39745 |
| [1 micro- | | | | | |
| grams/ml] | | | | | |

### Example 3

MIC of Novispirin G10 on Propionebacterium acnes

Minimal Inhibitory Concentration (MIC) of Novispirin G10 (SEQ ID NO: 16) was evaluated against P. acnes. Two strains were tested—one of which is clinically resistant to clindamycin and erythromycin.

The results shown in the table below emphasize the potent activity of novispirin G10 with all MIC activities being very low, including the activity against erythromycin- and clindamycin-resistant strains.

| | P. acnes | P. acnes |
|---|---|---|
| | (erythromycin sensitive) | (erythromycin resistant) |
| Novispirin G10 | 2 | 1 |
| Erythromycin | 0.25 | 64 |
| Clindamycin | 0.12 | 64 |

1. A method for treating a lung or skin infection or for promoting wound healing, comprising administering a human with a pharmaceutical composition comprising (a) an antimicrobial polypeptide comprises the sequence

| | |
|---|---|
| KNLRRX₁X₂RKX₃X₄HIIKKYG; | (SEQ ID NO:1) |

wherein X₁, X₂, X₃ and X₄ are independently selected from the group consisting of glycine, threonine, serine, glutamic acid, aspartic acid, isoleucine, D-alanine and D-isoleucine, provided that not more than 3 of the X residues are isoleucine; and (b) a pharmaceutical acceptable carrier.

2. The method of claim 1, wherein X₁, X₂, X₃ and X₄ are independently selected from the group consisting of glycine, threonine, serine, and isoleucine, provided that not more than 3 of the X residues are isoleucine.

3. The method of claim 2, wherein only one of X₁, X₂, X₃ and X₄ is selected from glycine, serine and threonine.

4. The method of claim 1, wherein the antimicrobial polypeptide comprises the amino acid sequence of any one of SEQ ID NO: 2 to SEQ ID NO: 29.

5. The method of claim 4, wherein the antimicrobial polypeptide comprises the amino acid sequence of SEQ ID NO: 16.

5. The method of claim 1, wherein the antimicrobial polypeptide consists essentially of the amino acid sequence of any one of SEQ ID NO: 2 to SEQ ID NO: 29.

6. The method of claim 1, wherein the carboxy terminus of the antimicrobial polypeptide is amidated.

7. The method of claim 1, wherein the pharmaceutical acceptable carrier comprises a chelating agent.

8. The method of claim 7, wherein the chelating agent is citrate.

9. The use of claim 1, wherein the pharmaceutical composition further comprises a second antimicrobial agent.

10. The method of claim 9, wherein the second antimicrobial agent is an antibiotic.

11. The method of claim 1, wherein the pharmaceutical composition is suitable for aerosol delivery of the antimicrobial peptide.

12. The method of claim 1, which is for the treatment of acne or atopic dermatitis or seborrheic dermatitis.

13. The method of claim 1, which is for the treatment of infections caused by Staphylococcus epidermidis, Staphylococcus aureus, Propionibacterium acnes, Pityrosporum ovale or Malassezia furfur.

14. The method of claim 1, which is for the treatment of pneumonia.

15. The method of claim 1, which is for promoting wound healing.
